(19) United States
(12) Patent Application Publication
(10) Pub. No.: US 2012/0184879 A1
(43) Pub. Date: Jul. 19, 2012

(54) MEDICAL DEVICE WITH TEMPERATURE SENSOR

(75) Inventors: Kevin J. Tanis, Collierville, TN (US); Debra Ann Arrington, Arlington, TN (US)

(73) Assignee: Smith & Nephew, Inc., Memphis, TN (US)

(21) Appl. No.: 13/278,582

(22) Filed: Oct. 21, 2011

Related U.S. Application Data
(60) Provisional application No. 61/432,691, filed on Jan. 14, 2011.
(60) Provisional application No. 61/471,946, filed on Apr. 5, 2011.

(51) Int. Cl. A61N 7/00

(52) U.S. Cl. 601/2

(57) ABSTRACT

A medical device includes at least one ultrasound transducer, at least one temperature sensor, at least one driver circuit, and at least one processing device. The temperature sensor is configured to detect a temperature at or adjacent a surface of the ultrasound transducer that emits ultrasound during operation of the ultrasound transducer. The driver circuit is coupled to the ultrasound transducer. The processing device is configured to (i) determine whether the temperature detected by the temperature sensor meets a defined condition, (ii) control the driver circuit such that the ultrasound transducer produces ultrasound with therapeutic properties if the determination indicates that the temperature meets the defined condition and (iii) control the driver circuit such that the ultrasound transducer does not produce ultrasound if the determination indicates that the temperature does not meet the defined condition.

### CROSS-REFERENCE TO RELATED APPLICATIONS

This application claims priority to and the full benefit of U.S. Provisional Application Ser. No. 61/432,691, filed Jan. 14, 2011, and titled “Ultrasound Medical Device With Temperature Sensor,” and U.S. Provisional Application Ser. No. 61/471,946, filed Apr. 5, 2011, and titled “Ultrasound Medical Device With Temperature Sensor,” the entire contents of which are incorporated herein by reference.

### TECHNICAL FIELD

This description relates to a medical device.

### BACKGROUND

For some conditions, a patient may benefit from treatment with a medical device, and treatment may be provided outside of a medical facility. Treatment that does not require supervision of a physician or travel to a medical facility can be convenient for a patient.

### SUMMARY

In a general aspect, a medical device includes at least one ultrasound transducer, at least one temperature sensor, at least one driver circuit, and at least one processing device. The temperature sensor is configured to detect a temperature at or adjacent a patient contact area of the medical device. The driver circuit is coupled to the ultrasound transducer. The processing device is configured to (i) determine whether the temperature detected by the temperature sensor meets a defined condition, (ii) control the driver circuit such that the ultrasound transducer produces ultrasound with therapeutic properties if the determination indicates that the temperature meets the defined condition and (iii) control the driver circuit such that the ultrasound transducer does not produce ultrasound if the determination indicates that the temperature does not meet the defined condition.

Implementations may include one or more of the following features. For example, the patient contact area is a surface of the ultrasound transducer that emits ultrasound during operation of the ultrasound transducer. The defined condition may be a minimum temperature. To determine whether the temperature detected by the temperature sensor meets the defined condition, the at least one processing device may be configured to determine whether the temperature meets or exceeds the minimum temperature. The defined condition may be a minimum rate of change of temperature. To determine whether the temperature detected by the temperature sensor meets the defined condition, the at least one processing device may be configured to determine whether the temperature is changing at or above the minimum rate of change of temperature. The defined condition may be a minimum rate of change of temperature and a minimum temperature. To determine whether the temperature detected by the temperature sensor meets the defined condition, the at least one processing device may be configured to determine both whether the temperature is changing at or above the minimum rate of change of temperature and whether the temperature meets or exceeds the minimum temperature.

The at least one processing device may be configured to (i) determine, while the driver circuit is controlled such that the ultrasound transducer produces ultrasound with therapeutic properties, whether the ultrasound has been produced for a defined time period, (ii) continue controlling the driver circuit such that the ultrasound transducer produces ultrasound with therapeutic properties if the determination indicates that the ultrasound has not been produced for a defined time period and (iii) control the driver circuit such that the ultrasound transducer does not produce ultrasound if the determination indicates that the ultrasound has been produced for a defined time period. The defined time may be a treatment duration.

The ultrasound transducer may include a matching layer that defines the surface that emits ultrasound during operation of the ultrasound transducer. The matching layer may define a hole and the temperature sensor may be disposed in the hole such that the temperature sensor detects the temperature at the surface of the ultrasound transducer that emits ultrasound during operation of the ultrasound transducer. The temperature sensor may be configured to produce a temperature signal that reflects the temperature at the surface.

The medical device may include a filter configured to receive the temperature signal and remove noise from the temperature signal to generate a filtered temperature signal. The medical device may also include an amplifier configured to receive the filtered temperature signal and amplify the filtered temperature signal to generate an amplified temperature signal that is provided to the at least one processing device. The temperature sensor may be a thermocouple or the temperature sensor may be a laser or optical based sensor.

The ultrasound transducer may include a piezoelectric transducer element that has an impedance that changes when a gel is applied to the surface that emits ultrasound during operation of the ultrasound transducer. The at least one processing device may be configured to (i) determine whether a gel has been applied to the surface based on the impedance of the piezoelectric transducer element and (ii) control the driver circuit such that the ultrasound transducer does not produce ultrasound if the gel has not been applied.

In another general aspect, a method of operating a medical device includes detecting a temperature at or adjacent a patient contact area of the medical device; determining that the detected temperature meets a defined condition; in response to determining that the detected temperature meets the defined condition, controlling a driver circuit coupled to the ultrasound transducer such that the ultrasound transducer produces ultrasound with therapeutic properties.

Implementations may include one or more of the following features. For example, detecting the temperature at or adjacent a patent contact area of the medical device includes detecting a temperature at or adjacent a surface of an ultrasound transducer, the surface emitting ultrasound during operation of the ultrasound transducer. The defined condition may be a minimum temperature. Determining whether the detected temperature meets a defined condition may include determining whether the temperature meets or exceeds the minimum temperature. The defined condition may be a minimum rate of change of temperature. Determining whether the detected temperature meets a defined condition may include determining whether the temperature is changing at or above the minimum rate of change of temperature. The defined condition may be a minimum rate of change of temperature and a minimum temperature. Determining whether the detected temperature meets a defined condition may include determining both whether the temperature is changing at or above the minimum rate of change of temperature and whether the temperature meets or exceeds the minimum temperature.

The method may include determining, while the driver circuit is controlled such that the ultrasound transducer produces ultrasound with therapeutic properties, whether the ultrasound has been produced for a defined time period; if the determination indicates that the ultrasound has not been produced for a defined time period, continuing to control the driver circuit such that the ultrasound transducer produces ultrasound with therapeutic properties; and if the determination indicates that the ultrasound has been produced for a defined time period, controlling the driver circuit such that the ultrasound transducer does not produce ultrasound. The defined time may be a treatment duration.

The method may include producing a temperature signal that reflects the temperature at the surface; removing noise from the temperature signal to generate a filtered temperature signal; and amplifying the filtered temperature signal to generate an amplified temperature signal. The method may include determining whether a gel has been applied to the surface based on an impedance of a piezoelectric transducer element and if the gel has not been applied, controlling the driver circuit such that the ultrasound transducer does not produce ultrasound.

In another general aspect a medical device includes: (i) at least one ultrasound transducer having a surface configured to emit ultrasound and (ii) means for detecting whether the surface of the transducer has been placed in contact with a portion of a body and controlling the ultrasound transducer such that that the surface emits ultrasound with therapeutic properties when the surface has been placed in contact with a portion of the body and does not produce ultrasound when the surface has not been placed in contact with a portion of the body.

In another general aspect, a medical device includes a treatment module configured to apply a medical treatment to a patient, the treatment module having a patient contact area configured to contact the patient and a temperature sensor configured to detect a temperature at or adjacent the patient contact area. The medical device includes at least one processing device configured to: (i) determine whether the temperature detected by the temperature sensor meets a defined condition; (ii) if the determination indicates that the temperature meets the defined condition, control the treatment module to apply a medical treatment; and (ii) if the determination indicates that the temperature does not meet the defined condition, control the treatment module such that the treatment module does not apply a medical treatment.

Implementations may include one or more of the following features. For example, the temperature sensor is configured to detect a temperature at or adjacent the patient contact area. The treatment device is configured to apply the medical treatment at the patient contact area.

In another general aspect, a medical device includes at least one ultrasound transducer, at least one temperature sensor, at least one driver circuit coupled to the ultrasound transducer, and at least one processing device. The temperature sensor is configured to detect a temperature at or adjacent a surface of the ultrasound transducer that emits ultrasound during operation of the ultrasound transducer. The at least one processing device is configured to determine whether the temperature detected by the temperature sensor meets a defined condition and to perform at least one of: (i) if the determination indicates that the temperature meets the defined condition, provide an indication that the temperature meets the defined condition; and (ii) if the determination indicates that the temperature does not meet the defined condition, provide an indication that the temperature does not meet the defined condition.

Implementations may include one or more of the following features. For example, the indication includes at least one of an audible alarm and a visible alarm. The at least one processing device is configured to control the driver circuit such that the ultrasound transducer produces ultrasound with therapeutic properties if the determination indicates that the temperature meets the defined condition. The at least one processing device is configured to control the driver circuit such that the ultrasound transducer does not produce ultrasound if the determination indicates that the temperature does not meet the defined condition.

The details of one or more implementations are set forth in the accompanying drawings and the description below. Other features and advantages will become apparent from the description, the drawings, and the claims.

### DESCRIPTION OF DRAWINGS

FIG. 1 is a perspective view of an example of a medical device.

FIG. 2 is a block diagram of the medical device.

FIGS. 3A and 3B are diagrams of a cross-section and bottom face, respectively, of a transducer of the medical device.

FIG. 4 is a flow diagram of an example of a process for administering medical treatments.

### DETAILED DESCRIPTION

In some implementations, a medical device includes a treatment module that is designed to be placed in contact with a portion of a patient's body in order for a treatment to be applied to the patient. The medical device also includes a mechanism configured to detect whether the treatment module is in contact with a portion of a patient's body, and to control the treatment module such that the treatment is applied when the treatment module is in contact with a portion of the patient's body and is not applied when the treatment module is not in contact with a portion of the patient's body.

For example, in one implementation, the treatment module includes an ultrasound transducer configured to emit ultrasound with therapeutic properties. The transducer includes a surface that is designed to be in contact with a portion of the patient's body for treatment. In such an implementation, the temperature at or adjacent the surface can be used to detect whether the surface is in contact with the patient's body. Contact with the patient's body will typically cause the temperature at the surface to increase to a certain temperature range. A temperature sensor is coupled to the surface and provides an indication of the temperature at a patient contact area of the medical device. The temperature sensor can be disposed, for example, at or adjacent the surface of the transducer that contacts the patient. A processing device receives the indication, and determines whether the detected temperature meets a defined condition (for example, temperature is at least equal to or higher than a defined threshold, and/or equal to or lower than a defined threshold). If the temperature meets the defined condition, the processing device causes the ultrasound transducer to produce the ultrasound. On the other hand, if the temperature does not meet the defined condition, the processing device does not cause the ultrasound transducer to produce the ultrasound.

In some implementations, if the temperature does not meet the defined condition, the processing device causes an indication to be presented to the user. For example, the processing device can cause an audible or visual warning to be presented to alert the patient that the defined condition is not met. The warning can indicate, for example, that the medical device is not ready to use. The warning or alarm can be provided in addition to, or as an alternative to, not causing the ultrasound transducer to produce the ultrasound.

In some implementations, if the temperature does meet the defined condition, the processing device causes an indication to be presented to the user. For example, the processing device can indicate that the temperature meets the defined condition on a display, which can indicate that the medical device is ready to be used. The indication can be provided in addition to, or as an alternative to, causing the ultrasound transducer to produce the ultrasound.

Some implementations of the medical device may provide the following advantages. For example, the medical device may be designed to provide only a certain number of treatments. By checking whether the ultrasound transducer is actually in contact with the human body before the treatment is applied, wastage of the pre-authorized treatments may be avoided. This would be very useful in instances where the treatments are expensive. In addition, by providing treatments only when the transducer is in contact with the human body, accidental misuse of the medical device can be deterred, which might happen for example if the patient turns on the device by mistake when the device is not contact with the patient's body.

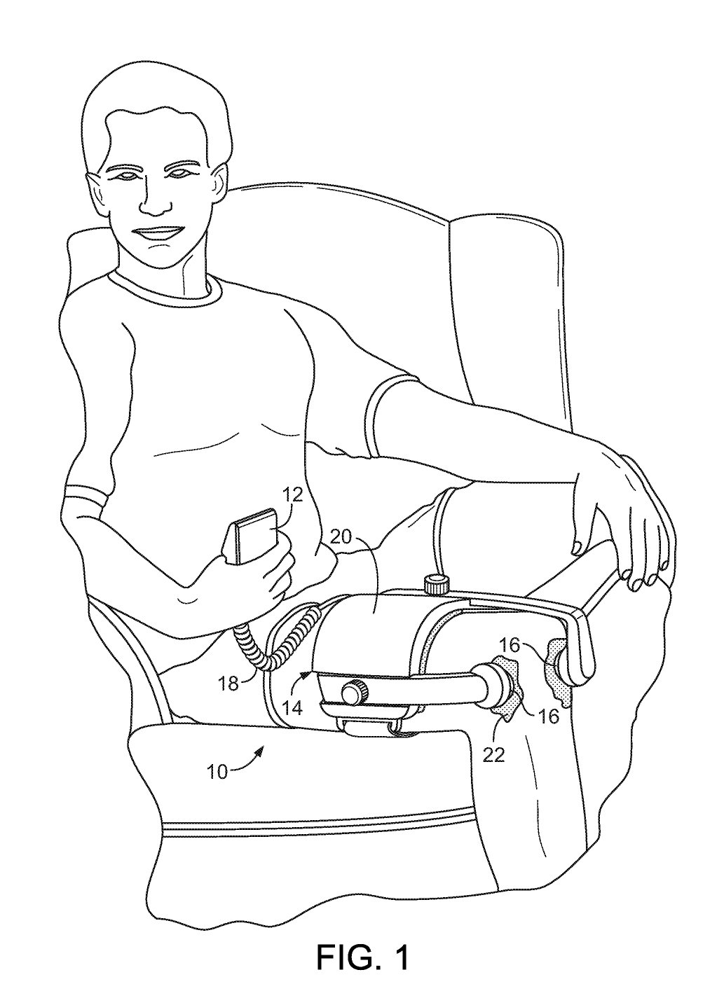

Referring to FIG. 1, a patient is shown using a medical device 10 that includes a treatment module for applying a treatment to the patient. In the examples illustrated, the medical device 10 is a portable ultrasonic treatment device that is equipped to provide treatments at the patient's convenience. The treatment module may include, for example, one or more ultrasound transducers 16 and at least one driver circuit coupled to the ultrasound transducers 16.

The medical device 10 can include a control unit 12 that controls the operation of the transducers 16. The control unit 12 can include the transducer driver circuit. The medical device 10 can also include cables 18 that can carry power, data, and control signals between the control unit 12 and the transducers 16.

The medical device 10 can include a placement module 14 that couples the transducers 16 at a location of the patient's body where treatment is needed, for example, over a fractured bone or next to damaged connective tissue. The placement module 14 can include a band, sleeve, or other connector 20 to fasten the one or more transducers to a treatment site. An ultrasound conducting gel 22 can be applied to the skin of the patient to enable the ultrasound to propagate effectively to the patient's tissue.

The medical device 10 can use low intensity, ultra high-frequency acoustic energy (ultrasound) to treat injuries, defects, or pathologies. For instance, the medical device 10 can be designed to treat injuries, defects, or pathologies of bones or connective tissue, and, in some instances, can increase vascularization of ischemic or grafted tissue. The medical device 10 may be used as an adjunct to surgical repair, in order to speed healing, or in some cases can be used alone to heal tissue injuries without surgery (e.g., for degenerative diseases such as osteoarthritis, tendonosis, and tendonitis). The medical device 10 can be suitable for use in treatment of bone fractures and/or connective tissues associated with joints, such as those in the hand, foot, wrist, ankle, knee, elbow, hip, shoulder, back, and neck.

For example, following surgery, the medical device 10 can be applied non-invasively to the outside of the body (e.g., coupled to the skin with coupling media, such as a gel) in the region of the repaired tissue. The medical device 10 can be operated to transmit ultrasound (for example, in the form of pulses) into the tissue in need of treatment, or at the interface with the uninjured tissues. Exposure to the ultrasound can stimulate a faster, better quality repair of the tissue. At a bone interface, the ultrasound can also stimulate bone repair and bone ingrowth into repair or graft tissue. This can give rise to a faster, stronger repair and improved integration of the interface between, for example, tendon, ligament, and bone. The medical device 10 may also be used to non-invasively treat pathologies of connective tissues, such as osteoarthritis, ligament and tendon conditions, without the need for a surgical procedure.

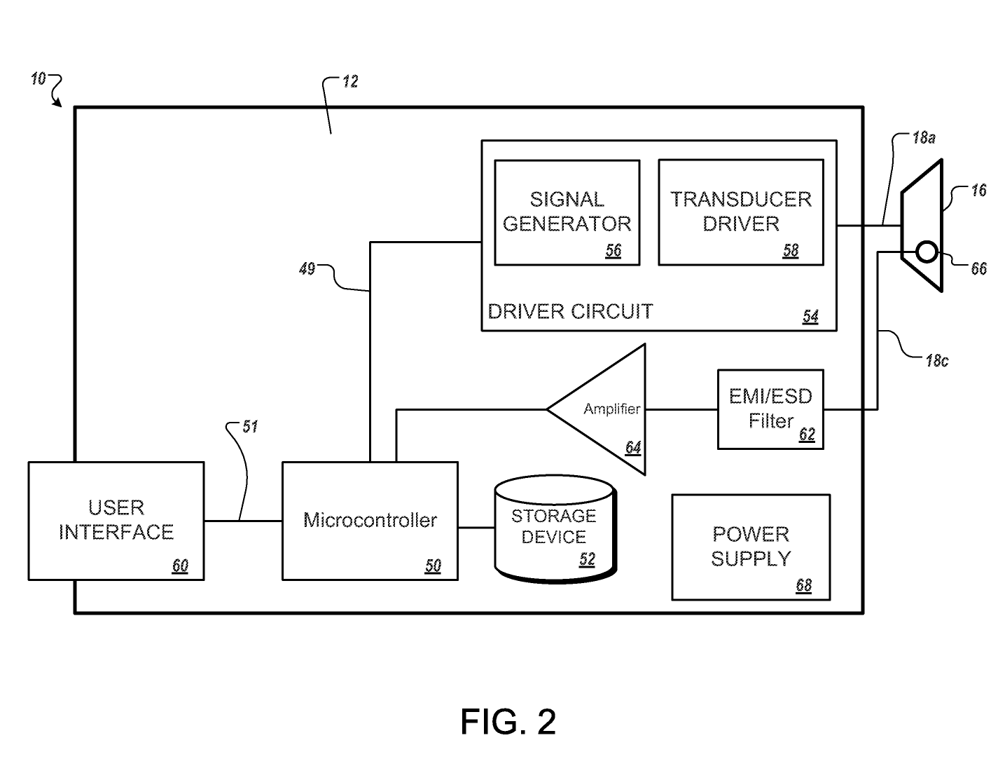

Referring to FIG. 2, in one implementation, the control unit 12 of the medical device 10 includes a processing device, such as a microcontroller 50, which executes instructions stored on a storage device 52. The control unit 12 also includes a power supply 68 that powers the various components of the control unit 12, a user interface 60, an EMI/ESD filter 62, an amplifier 64, and a driver circuit 54 that drives the ultrasound transducer 16.

A temperature sensor 66 is embedded on the surface of the ultrasound transducer 16 that is to be placed in contact with the patient, and connected by a cable 18c to an input of the EMI/ESD filter 62. An output of the EMI/ESD filter 62 is attached to an input of the amplifier 64, which has an output connected to the microcontroller 50. The transducer 16 is attached to a driver circuit 54 via a cable 18a. The driver circuit 54 is connected to the microcontroller 50 through a control line 49 and includes a signal generator 56 and a transducer driver 58. The user interface 60 is attached to the microcontroller 50 via a separate cable 51.

The change in temperature of the surface of the ultrasound transducer is measured by the temperature sensor 66 and is propagated to the microcontroller 50 via the EMI/ESD filter 62 and the amplifier 64. In a particular implementation, the temperature sensor is a thermocouple, such as a J or K type thermocouple. J and K type thermocouples are created by a junction of two materials that outputs a known voltage at a specific temperature, and the voltage varies with temperature. The J and K types are passive varieties that give a detectable delta difference for the operational temperature range of the medical device 10. Alternatively, some other varieties of thermocouples might use active drivers, such as Wheatstone Bridge. Alternative implementations of the medical device 10 may use different temperature sensors (for example, laser or optical), which may use active drivers.

In some implementations, the temperature sensor 66 can use a semiconductor junction, for example, a junction of a diode or a bipolar junction transistor, to measure temperature. The semiconductor junction can be biased such that an amount electrical current through the junction indicates the temperature at the junction, or such that a change in current indicates a change in temperature. As an example, the temperature sensor 66 can be a diode-connected bipolar junction transistor. As an example, an NPN transistor can be used with the collector terminal and the base terminal connected together. A current source can be connected to the base and collector, and a current sink can be connected to the emitter terminal. With an appropriate voltage across the transistor, as the temperature at the transistor changes, the flow of electrical current through the transistor changes in response. For example, an increase temperature can increase the current flowing through the transistor. The current through the transistor can be detected and used to determine the temperature at or near the transistor.

Other implementations may employ other types of temperature sensors such as resistance temperature measurement devices, both metallic and ceramic (including thermistors), infrared temperature measurement devices, fluid expansion temperature measurement devices, bimetallic temperature measurement devices, or change-in-state temperature measurement devices.

Resistance temperature measurement devices capitalize on the fact that the electrical resistance of a material changes as its temperature changes. With such devices, the resistance is measured and translated into a temperature. Infrared temperature measurement devices measure temperature by measuring the thermal radiation emitted by a material. Fluid expansion temperature measurement devices exploit the thermal expansion of a fluid based on temperature. The expansion of the fluid (whether liquid or gas) can be determined and translated into a temperature. Bimetallic temperature measurement devices take advantage of the difference in rate of thermal expansion between different metals. Two metals are bonded together such that, when heated, one side will expand more than the other, and the resulting bending is translated into a temperature. Change-of-state temperature measurement devices typically involve a material whose appearance changes based on temperature.

Thermocouples (and other temperature sensors) may produce voltages in the millivolt range, and the temperature signal from the thermocouples may contain ambient noise. The EMI/ESD filter 62 removes noise from the temperature signal, and the amplifier amplifies the filtered temperature signal to produce an amplified signal with a range appropriate for the processing device 50, such as the 3 to 5 volt range

The microcontroller 50 reads the temperature detected by the temperature sensor 66 and determines whether the temperature meets a defined condition. If the determination indicates that the defined condition is met, the microcontroller 50 can control the treatment module (for example, driver circuit 54 and transducers 16) to apply the treatment. If the determination indicates that the defined condition is not satisfied, then the microcontroller 50 can control the treatment module such that the treatment is not applied. In one implementation, the defined condition is a minimum temperature on the surface of the ultrasound transducer 16, as measured by the temperature sensor 66. Alternatively, or additionally, the defined condition may be a particular rate of change of temperature on the surface of the ultrasound transducer 16, as measured by the temperature sensor 66. Measuring the rate of change of temperature can be useful, for example, if the temperature does not rise quickly enough to allow the patient to have a timely treatment. Furthermore, the defined condition may be a particular rate of change of the temperature and a minimum temperature. The minimum temperature or the rate of change of temperature (or both) may be indicative of whether the transducer surface is completely in contact with the skin.

In some implementations, the medical device indicates to the user whether the temperature meets the defined condition. The medical device can provide an indication when the temperature meets the defined condition, provide an indication when the temperature does not meet the defined condition, or provide both indications. The indication can be provided on, for example, a display, a light, or another user interface element, or can be provided audibly.

When the microcontroller 50 reads the temperature detected by the temperature sensor 66, the microcontroller 50 can store the detected temperature on the storage device 52. The microcontroller 50 can maintain a temperature log that indicates detected temperatures and corresponding times that the temperatures were detected. The microcontroller 50 can transfer the temperature log to another system and may display information about the detected temperatures on a screen of the medical device 10, for example, as a chart or graph showing recorded temperatures over time.

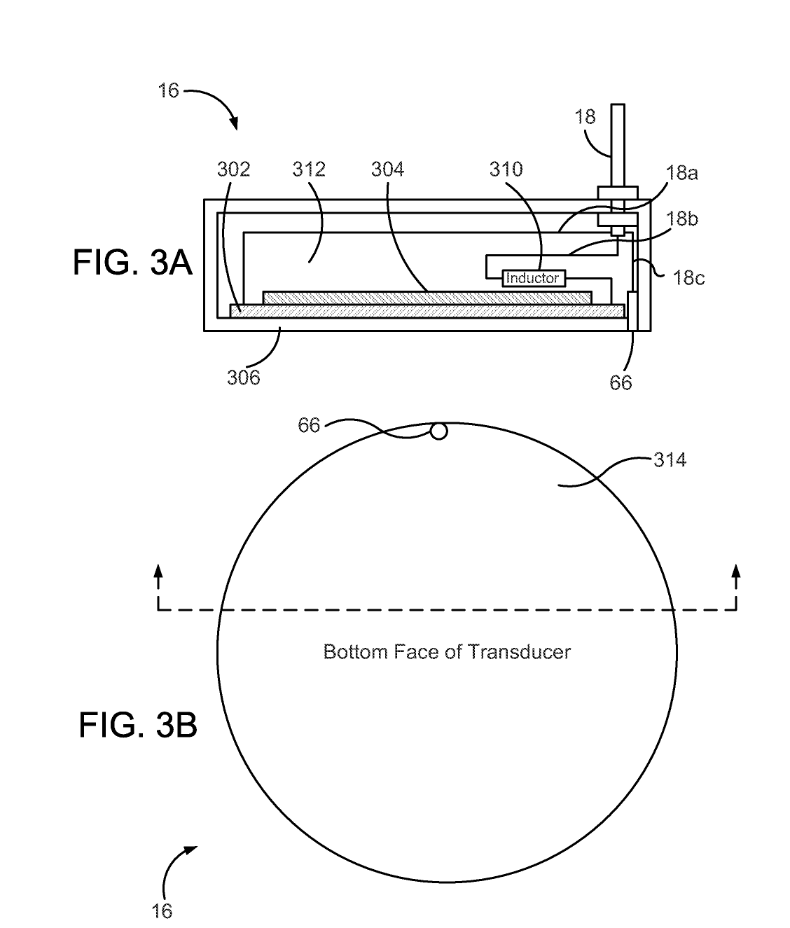

In some implementations, the microcontroller 50 may determine whether an ultrasound conducting gel 22 is applied to the surface of the ultrasound transducer 16 and control the driver circuit 54 appropriately. For example, the microcontroller 50 determine whether the gel 22 is applied by detecting the impedance of the transducing component of the transducer 16 (for example, a piezoelectric transducer, as described in FIG. 3), and determining whether the impedance is within a particular range. If the gel 22 has been applied, the microcontroller 50 controls the driver circuit 54 such that the treatment is applied and, if the gel 22 has not been applied, the microcontroller 50 controls the driver circuit 54 such that the treatment is not applied.

Applying the treatment can include controlling the driver circuit 54 to produce ultrasound with therapeutic properties. Controlling the driver circuit 54 to produce ultrasound can include, for example, activating the driver circuit 54 by supplying power to the driver circuit 54, sending control signals to the driver circuit 54, or causing the driver circuit 54 to produce a particular output. Not applying the treatment can include the microcontroller 50 controlling the driver circuit 54 such that ultrasound with therapeutic properties is not produced. Controlling the driver circuit so that the treatment is not applied can include not activating the driver circuit 54, deactivating the driver circuit 54, re-setting the output of the driver circuit 54 (for example, setting the amplitude to zero), and/or otherwise limiting or preventing treatment. The microcontroller 50 can also be configured to control other components described below, for example, through instructions stored on the storage device 52.

The driver circuit 54 can be configured to send drive signals that cause the transducer 16 to generate ultrasound with therapeutic properties. For example, the signal generator 56 can generate a signal and the transducer driver 58 can drive the transducers 16 according to the generated signal. In an implementation, the ultrasound generated by the transducers 16 can include low intensity ultrasound (for example, less than 100 mW/cm², such as 30 mW/cm²) having a frequency ranging approximately between 1 and 2 MHz, more particularly about 1.5 MHz. The ultrasound can be pulsed, with a pulse width ranging from about 10 to 2,000 microseconds, more particularly about 200 microseconds, with a repetition frequency ranging from about 0.1 to about 10 KHz, more particularly about 1 KHz.

The storage device 52 can store a device identifier that identifies the particular medical device 10. The device identifier can uniquely identify the medical device 10 and distinguish it from all other ultrasonic treatment devices, even those of the same type or model. The storage device 52 can also store information about the treatments that are authorized for the medical device 10, for example, a number of treatments that are authorized or an authorization code that authorizes treatments.

The user interface 60 can provide information to the patient and enable treatment to be initiated. The user interface 60 may include one or more input devices or controls, for example, buttons, a keypad, or a touch-sensitive screen. The user interface 60 may be used by a patient or other person, for example, to enter user input that indicates that a treatment should be administered by the medical device. The user interface 60 may also include output devices, for example a screen, a liquid crystal display, or lights. When the microcontroller 50 determines that treatment is not authorized, the microcontroller 50 can provide an indication to the patient on the user interface 60 that more treatments need to be authorized.

The power supply 68 can provide power to the components of the medical device 10, including the driver circuit 54, the microcontroller 50, the storage device 52, the EMI/ESD filter 62, the amplifier 64, and the user interface 60.

In one implementation, to apply a treatment, the surface of the transducer 16 is coated with a thin layer of a particular ultrasound conducting gel 22 and the arm of the medical device 10 is placed such that the transducer surface is in contact with the human body. When the medical device 10 is powered on by the patient, the control unit 12 performs a self-test to ensure that the device configuration is consistent with the pre-programmed configuration for applying the treatment. The physical contact of the gel-coated transducer surface 16 with the human body may cause a rise in the temperature of the transducer surface 16 which can be detected by the temperature sensor 66 and transmitted to the microcontroller 50. The application of the gel 22 on the transducer surface 16 may also cause a change in impedance of the transducing component, the measurement of which can be transmitted to the microcontroller 50. The microcontroller 50 may obtain readings for both the change in temperature and the change in impedance and check the measured values against predetermined ranges stored in memory to decide whether the conditions for the application of the treatment are met. For example, the check may be to see whether the temperature is equal to or higher than a threshold temperature, which may be, for example, 85 degrees Fahrenheit in a particular implementation. The check may also include an upper bound on the temperature (for example, also check if the temperature is below 100 degrees Fahrenheit in case the treatment should not be applied when a fever is present). As an alternative or in addition to the above, the microcontroller 50 may check for a rate of change of temperature (for example, 0.1 degrees Fahrenheit per second) before allowing the treatment to be applied.

If the physical conditions are met, the microcontroller 50 controls the driver circuit 54 to apply the treatment for a pre-determined period of time. The microcontroller 50 will control the medical device such that the treatment is not applied if the physical conditions are not met, for example, if the temperature of the transducer surface 16 is not within the expected range. The latter may happen, for example, if the transducer surface 16 is not in contact with the human body and therefore the temperature sensor 66 does not detect a change in temperature, even if the change in impedance is within the expected range. This might be the case, for example, if the patient applied the gel 22 and then placed the medical device 10 on the table and turned it on. The microcontroller 50 may also control the medical device 10 such that the treatment is not applied if the microcontroller 50 determines that the treatment has been applied for the pre-determined period of time. In addition, or as an alternative, the microcontroller 50 controls a display, speaker, or other output device to provide a warning or alarm to a patient when the detected temperature or detected temperature change is not within the expected range.

FIGS. 3A and 3B are diagrams of a cross-section and bottom view, respectively, of an example of the transducer 16. The transducer 16 includes a piezoelectric (PZT) ceramic element 302, which is backed by isolation foam 304. The PZT ceramic 302 and the isolation foam 304 are placed in the housing 312, which may be filled with an epoxy. Also present in the housing is an inductor 310. A matching layer 306 covers the PZT, and defines the surface 314 that emits ultrasound during operation of the transducer 16. The matching layer 306 defines a hole in which the temperature sensor 66 is disposed such that the temperature sensor 66 detects the temperature at or adjacent the surface 314. The PZT ceramic 302, the inductor 310 and the temperature sensor 66 are connected to the controller circuit 12 through cables 18a, 18b and 18c respectively, which are aggregated together into cable 18.

The matching layer 306, which includes the transducer surface 314, helps to reduce the impedance to the ultrasound so that the ultrasound propagates outward. The matching layer 306 is set to a thickness, for example, that is about one-quarter of the expected wavelength of the ultrasound. The matching layer 306 vibrates homogenously with the PZT ceramic 302, which transduces the electric signal to an acoustic signal. The isolation foam 304 prevents or reduces the possibility of the ultrasound signal from going backward inside the housing 312. For instance, air pockets in the foam prevent the signal from propagating into the housing 312.

The treatment cable 18 is used to cause the PZT ceramic 302 to generate ultrasound. For instance, an AC waveform with ground is applied to the PZT ceramic 302 via the cables 18a and 18b. The positive side applies the AC waveform, while the other side is ground. The inductor 310 is included in series with the PZT 302 and is used, for example, to achieve a 50 ohm load with zero phase shift. In other words, the inductor 310 is used to provide phase matching with the capacitance of the PZT ceramic 302. The impedance of the PZT 302 may change when a gel is applied to the surface 314. As described above, this change in impedance may be used to detect whether or not the gel has been applied.

Alternatively or additionally, the temperature sensor 66 may be attached to the surface 314 of the transducer 16 rather than passing through the hole in the matching layer 306.

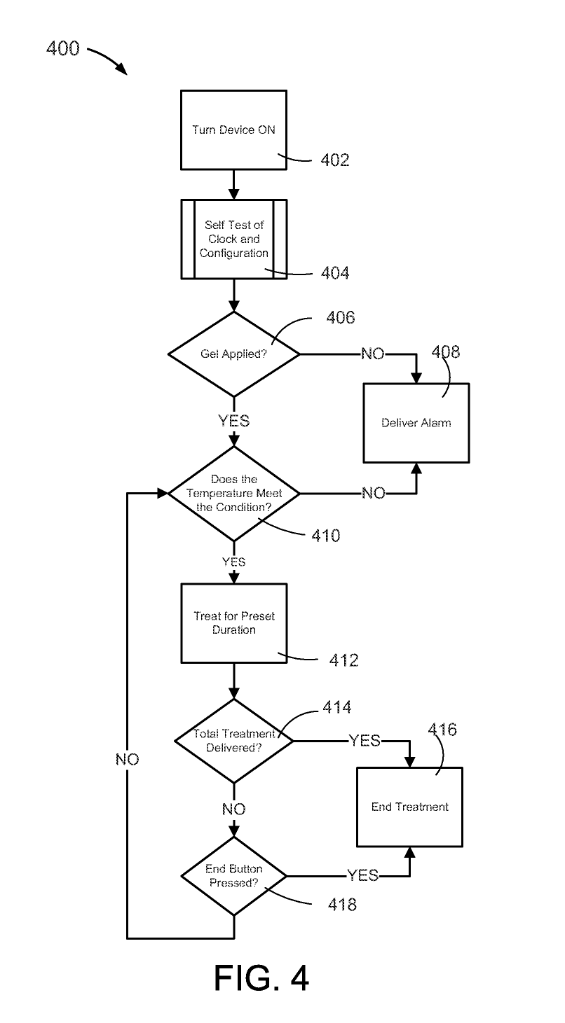

FIG. 4 is a flowchart illustrating a process 400 for application of a medical treatment by a medical device. The medical device can be an ultrasonic treatment device such as medical device 10, and will be described with respect to an implementation of medical device 10 that determines whether gel has been applied to the bottom surface 314 of the transducer 16 and whether a temperature of the bottom surface 314 meets a defined condition. The actions performed by the medical device can be performed by one or more processing devices of the medical device configured to perform those actions.

A patient may attempt to initiate treatment with the medical device 10 (402). For example, the patient can turn on the medical device and subsequently enter a user input indicating that a treatment should be administered by the medical device.

The medical device performs a series of internal tests to determine whether its clock is running correctly and whether all the mechanical parts like the ultrasound transducer 16 are properly connected and operational (404). The determination whether the medical device is correctly configured and operating normally can be performed in response to the attempt to initiate treatment in (402).

If the determination indicates that the medical device 10 is correctly configured and operational, the medical device determines whether the conditions are met to apply the treatment. For instance, the medical device determines whether the ultrasound conducting gel 22 is applied to the face of the ultrasound transducer 16 (406). The determination can be made, for example, by measuring the impedance of the PZT 302 and determining if the impedance is within a certain range that would occur when gel is applied to the surface 314. If the determination indicates that gel is not properly applied, the medical device delivers an alarm to alert the patient (408) and the treatment is not applied.

If the determination indicates that the gel is properly applied, the medical device determines whether the temperature at or adjacent the surface of the transducer 16 meets a defined condition (410). The condition may be, for example, that the temperature of the transducer surface be in the range that corresponds to the expected range that would occur when the transducer is coupled to the human body (for example, the temperature is above a minimum temperature, and/or below a maximum threshold). The determination can be made, for example, by measuring the temperature on the surface of the transducer as measured by the temperature sensor 66. Alternatively, or in addition to the above, the condition may be that the rate of change of temperature on the surface of the transducer meets a minimum rate of change. For example, the minimum rate of change condition can be met when the rate of change is at or above a minimum rate of change.

If the determination indicates that the temperature condition is not met, the medical device delivers an alarm to alert the patient (408). The medical device can also control a driver circuit 54 so that treatment is not applied, for example, by not activating the driver circuit so that the treatment is prevented. The medical device may determine that the temperature condition is not met, for example, if the temperature measured by the temperature sensor 66 is not within the expected range, or the rate of change of temperature measured by the temperature sensor is not consistent with an expected rate of change of temperature. Either or both of the previous two determinations may be made, for example, if the transducer surface is not in contact with the human body.

If the determination indicates that the temperature condition is met, the medical device 10 controls a driver circuit to apply treatment for a preset duration (for example, 1 second) (412). For example, the medical device can control an ultrasound transducer driver circuit 54 in a manner that causes one or more ultrasound transducers to produce ultrasound with therapeutic properties. The driver circuit may continue to drive the ultrasound transducers until treatment is delivered for the preset duration (for example, one second).

When the preset duration is over, the medical device 10 determines if the treatment has been delivered for the treatment duration (for example, 20 minutes) (414). When the determination indicates that the treatment duration has been completed, the medical device deactivates the driver circuit so that the treatment is halted (416). If the determination indicates that the treatment duration has not been completed, the medical device 10 determines whether an end button is pressed (418). The determination can be made, for example, if the medical device receives a signal which indicates that the end button is pressed. When the determination indicates that the end button has been pressed, the driver circuit can be deactivated so that the treatment is finished (416).

If the end button has not been pushed, the medical device 10 determines whether the temperature condition continues to be met, as described previously (410). If the determination indicates that the temperature condition is not met, the medical device delivers an alarm to alert the patient and the treatment is stopped (408). The medical device also deactivates the driver circuit so that the treatment is not delivered. If the temperature condition continues to be met, the driver circuit continues to drive the ultrasound transducers such that therapeutic ultrasound is produced. This continues until the treatment duration is met (414), the stop button is pushed (418), or the temperature condition is no longer met (410).

An alternative implementation of the medical device may include a permanent gel on the surface of the transducer. In such an implementation, the condition for applying the treatment may only include the determination whether the temperature condition is met.

Another alternative implementation of the medical device may have multiple transducers. Each transducer may have multiple temperature sensors embedded on the face of the transducer. This may be the case, for example, if the transducer is differently shaped with a larger surface area, in which case multiple temperature sensors may be useful to determine whether the entire transducer surface is in contact with the human body. In such an alternative implementation, the medical device circuitry can be configured such that it would separately check the temperature measured by each temperature sensor and, for example, prevent the application of the treatment if even one of measurements is not in range.

The techniques described above are not limited to any particular hardware or software configuration. Rather, they may be implemented using hardware, software, or a combination of both. The methods and processes described may be implemented as computer programs that are executed on programmable computers comprising at least one processor and at least one data storage system. The programs may be implemented in a high-level programming language and may also be implemented in assembly or other lower level languages, if desired.

Any such program will typically be stored on a computer-usable storage medium or device (e.g., CD-ROM, RAM, or magnetic disk). When read into the processor of the computer and executed, the instructions of the program cause the programmable computer to carry out the various operations described above.

A number of implementations have been described. Nevertheless, it will be understood that various modifications may be made. For example, the techniques described above can be implemented for medical devices that provide medical treatments other than ultrasound treatments. A temperature sensor may be located at any patient contact surface of a medical device, or at any other location that permits measurement of a temperature at a site to be treated. As described above, alarms can be provided and treatment can be limited when a detected temperature does not meet a defined condition, even if the treatment is not an ultrasound treatment. Treatment can be applied when the defined condition is met. Accordingly, other implementations are within the scope of the following claims.

1. A medical device comprising:
at least one ultrasound transducer;
at least one driver circuit coupled to the ultrasound transducer;
at least one temperature sensor configured to detect a temperature at or adjacent a patient contact area of the medical device;
at least one processing device configured to perform the following:
determine whether the temperature detected by the temperature sensor meets a defined condition;
if the determination indicates that the temperature meets the defined condition, control the driver circuit such that the ultrasound transducer produces ultrasound with therapeutic properties; and
if the determination indicates that the temperature does not meet the defined condition, control the driver circuit such that the ultrasound transducer does not produce ultrasound.

2. The medical device of claim 1 wherein the patient contact area is a surface of the ultrasound transducer that emits ultrasound during operation of the ultrasound transducer.

3. The medical device of claim 1 wherein:
the defined condition is a minimum temperature; and
to determine whether the temperature detected by the temperature sensor meets the defined condition, the at least one processing device is configured to determine whether the temperature meets or exceeds the minimum temperature.

4. The medical device of claim 1 wherein:
the defined condition is a minimum rate of change of temperature; and
to determine whether the temperature detected by the temperature sensor meets the defined condition, the at least one processing device is configured to determine whether the temperature is changing at or above the minimum rate of change of temperature.

5. The medical device of claim 1 wherein:
the defined condition is a minimum rate of change of temperature and a minimum temperature; and
to determine whether the temperature detected by the temperature sensor meets the defined condition, the at least one processing device is configured to determine both whether the temperature is changing at or above the minimum rate of change of temperature and whether the temperature meets or exceeds the minimum temperature.

6. The medical device of claim 1 wherein at least one processing device is configured to perform the following:
determine, while the driver circuit is controlled such that the ultrasound transducer produces ultrasound with therapeutic properties, whether the ultrasound has been produced for a defined time period;
if the determination indicates that the ultrasound has not been produced for a defined time period, continue controlling the driver circuit such that the ultrasound transducer produces ultrasound with therapeutic properties; and
if the determination indicates that the ultrasound has been produced for a defined time period, control the driver circuit such that the ultrasound transducer does not produce ultrasound.

7. The medical device of claim 6 wherein the defined time is a treatment duration.

8. The medical device of claim 1 wherein:
the ultrasound transducer includes a matching layer that defines the surface that emits ultrasound during operation of the ultrasound transducer;
the matching layer defines a hole; and
the temperature sensor is disposed in the hole such that the temperature sensor detects the temperature at or adjacent the surface of the ultrasound transducer that emits ultrasound during operation of the ultrasound transducer.

9. The medical device of claim 1 wherein the temperature sensor is configured to produce a temperature signal that reflects the temperature at or adjacent the surface, the medical device comprising:
a filter configured to receive the temperature signal and remove noise from the temperature signal to generate a filtered temperature signal; and
an amplifier configured to receive the filtered temperature signal and amplify the filtered temperature signal to generate an amplified temperature signal that is provided to the at least one processing device.

10. The medical device of claim 1 wherein the temperature sensor is a thermocouple.

11. The medical device of claim 1 wherein the temperature sensor is a laser or optical based sensor.

12. The medical device of claim 1 wherein:
the ultrasound transducer includes a piezoelectric transducer element that has an impedance that changes when a gel is applied to the surface that emits ultrasound during operation of the ultrasound transducer;
the at least one processing device is configured to determine whether a gel has been applied to the surface based on the impedance of the piezoelectric transducer element and, if the gel has not been applied, control the driver circuit such that the ultrasound transducer does not produce ultrasound.

13. A method of operating a medical device comprising:
detecting a temperature at or adjacent a patient contact area of the medical device;
determining that the detected temperature meets a defined condition; and
in response to determining that the detected temperature meets the defined condition, controlling a driver circuit coupled to the ultrasound transducer such that the ultrasound transducer produces ultrasound with therapeutic properties.

14. The method of claim 13, wherein detecting the temperature at or adjacent a patient contact area of the medical device comprises detecting a temperature at or adjacent a surface of an ultrasound transducer, the surface emitting ultrasound during operation of the ultrasound transducer.

15. The method of claim 13 wherein:
the defined condition is a minimum temperature; and
determining whether the detected temperature meets a defined condition comprises determining whether the temperature meets or exceeds the minimum temperature.

16. The method of claim 13 wherein:
the defined condition is a minimum rate of change of temperature; and
determining whether the detected temperature meets a defined condition comprises determining whether the temperature is changing at or above the minimum rate of change of temperature.

17. The method of claim 13 wherein:
the defined condition is a minimum rate of change of temperature and a minimum temperature; and
determining whether the detected temperature meets a defined condition comprises determining both whether the temperature is changing at or above the minimum rate of change of temperature and whether the temperature meets or exceeds the minimum temperature.

18. The method of claim 13 comprising:
determining, while the driver circuit is controlled such that the ultrasound transducer produces ultrasound with therapeutic properties, whether the ultrasound has been produced for a defined time period;
if the determination indicates that the ultrasound has not been produced for a defined time period, continuing to control the driver circuit such that the ultrasound transducer produces ultrasound with therapeutic properties; and
if the determination indicates that the ultrasound has been produced for a defined time period, controlling the driver circuit such that the ultrasound transducer does not produce ultrasound.

19. The method of claim 18 wherein the defined time is a treatment duration.

20. The method of claim 13 comprising:
producing a temperature signal that reflects the temperature at or adjacent the surface;
removing noise from the temperature signal to generate a filtered temperature signal; and
amplifying the filtered temperature signal to generate an amplified temperature signal.

21. The method of claim 13 wherein:
determining whether a gel has been applied to the surface based on an impedance of a piezoelectric transducer element; and
if the gel has not been applied, controlling the driver circuit such that the ultrasound transducer does not produce ultrasound.

22. A medical device comprising:
at least one ultrasound transducer having a surface configured to emit ultrasound; and
means for detecting whether the surface of the transducer has been placed in contact with a portion of a body and controlling the ultrasound transducer such that that the surface emits ultrasound with therapeutic properties when the surface has been placed in contact with a portion of the body and does not produce ultrasound when the surface has not been placed in contact with a portion of the body.

23. A medical device comprising:
a treatment module configured to apply a medical treatment to a patient, the treatment module having a patient contact area configured to contact the patient;
a temperature sensor configured to detect a temperature at or adjacent the patient contact area;
at least one processing device configured to perform the following:
determine whether the temperature detected by the temperature sensor meets a defined condition;
if the determination indicates that the temperature meets the defined condition, control the treatment module to apply a medical treatment; and
if the determination indicates that the temperature does not meet the defined condition, control the treatment module such that the treatment module does not apply a medical treatment.

24. The medical device of claim 23 wherein the temperature sensor is configured to detect a temperature at or adjacent the patient contact area.

25. The medical device of claim 23 wherein the treatment device is configured to apply the medical treatment at the patient contact area.

26. A medical device comprising:
at least one ultrasound transducer;
at least one temperature sensor configured to detect a temperature at or adjacent a surface of the ultrasound transducer that emits ultrasound during operation of the ultrasound transducer;
at least one driver circuit coupled to the ultrasound transducer;
at least one processing device configured to perform the following:
determine whether the temperature detected by the temperature sensor meets a defined condition; and
perform at least one of:
(i) if the determination indicates that the temperature meets the defined condition, provide an indication that the temperature meets the defined condition; and
(ii) if the determination indicates that the temperature does not meet the defined condition, provide an indication that the temperature does not meet the defined condition.

27. The medical device of claim 26 wherein the indication includes at least one of an audible alarm or a visible alarm.
